(19) United States
(12) Patent Application Publication
(10) Pub. No.: US 2026/0249060 A1
(43) Pub. Date: Aug. 27, 2026

(54) EXPANDABLE DEVICE FOR A BODY CAVITY

(71) Applicant: AG IP HOLDING, LLC, Okemos, MI (US)

(72) Inventors: Patrick Kenneth Powell, Grosse Pointe Park, MI (US); Eun-Jung Kim, Grosse Pointe Park, MI (US)

(21) Appl. No.: 18/854,735

(22) PCT Filed: Apr. 11, 2023
(86) PCT No.: PCT/US2023/018124

§ 371 Date: Oct. 7, 2024

Related U.S. Application Data
(60) Provisional application No. 63/329,627, filed on Apr. 11, 2022.
(60) Provisional application No. 63/342,653, filed on May 17, 2022.
(60) Provisional application No. 63/345,930, filed on May 26, 2022.
(60) Provisional application No. 63/358,334, filed on Jul. 5, 2022.
(60) Provisional application No. 63/358,337, filed on Jul. 5, 2022.
(60) Provisional application No. 63/359,262, filed on Jul. 8, 2022.
(60) Provisional application No. 63/388,405, filed on Jul. 12, 2022.
(60) Provisional application No. 63/389,205, filed on Jul. 14, 2022.
(60) Provisional application No. 63/389,496, filed on Jul. 15, 2022.
(60) Provisional application No. 63/405,173, filed on Sep. 9, 2022.
(60) Provisional application No. 63/416,647, filed on Oct. 17, 2022.
(60) Provisional application No. 63/482,116, filed on Jan. 30, 2023.

(51) Int. Cl. A61M 31/00; A61M 1/00

(52) U.S. Cl. CPC A61M 31/002; A61M 1/71; A61M 2202/0007; A61M 2205/32; A61M 2205/3344; A61M 2205/8281

(57) ABSTRACT

A device includes a casing that has a collapsed state and an expanded state a spring device in the casing, an inlet port connected with the casing, and a breaker connected with the inlet port. The casing is evacuated to define a pressure differential that holds the casing in the collapsed state, and in the collapsed state the casing holds the spring device in a stressed state that has a stored potential energy. The inlet port is sealed to maintain the pressure differential. The breaker operable to open the inlet port and equalize the pressure differential, whereupon the casing releases the spring device from the stressed state to convert the stored potential energy to kinetic energy that moves the casing from the collapsed state to the expanded state.

### BACKGROUND

The ability to control the movement of substances through a body cavity is important in the field of medicine. A body cavity could include an arterial passage, a vagina, a rectal passage, a urinary tract, an ear, nose, or throat canal, or a wound cavity. For example, there may be a need to prevent leakage through the cavity, manage the flow through the cavity, reinforce the cavity while maintaining flow, or prevent ingress of fluid or noise through the cavity.

One type of device for controlling flow in a body cavity is a tampon. Most typically, a tampon is a cylindrical-shaped absorbent device made of cotton or other material that is inserted into the vagina during menstruation to absorb menstrual fluid and prevent it from leaking out of the body. Other types of devices for controlling flow include plugs or stoppers. For example, rectal plugs are typically made of silicone and are designed to be inserted into the rectum to prevent the passage of stool or other materials; nasal plugs are typically made of soft silicone or foam and are designed to be inserted into the nostrils to control bleeding or to prevent the passage of air or fluids; and ear plugs are typically made of foam or silicone and are designed to be inserted into the ear canal to block out noise or water.

Although such devices are often effective, some suffer drawbacks, such as (i) difficulty controlling expansion of the device to fill the cavity, (ii) difficulty to insert into the cavity and requiring a tool or robotic assistance, (iii) difficulty controlling absorption, (iv) unable to be rapidly inserted to quickly control flow through the cavity, (v) difficulty to remove from the cavity after use, (vi) difficulty conforming to the cavity, (vii) the device prevents tools or other objects from being passed through the device, or (viii) the weight of fluid filled plugs, such as a saline filled cavity plug.

### SUMMARY

A device according to an example of the present disclosure includes at least one casing that has a collapsed state and an expanded state, and at least one spring device in the at least one casing. The at least one casing is evacuated to define a pressure differential that holds the at least one casing in the collapsed state, and in the collapsed state the at least one casing holds the at least one spring device in a stressed state with a stored potential energy. At least one inlet port is connected with the at least one casing. A breaker is connected with the at least one inlet port. The breaker is operable to open the at least one inlet port and equalize the pressure differential, whereupon the at least one casing releases the at least one spring device from the stressed state to convert the stored potential energy to kinetic energy that moves the at least one casing from the collapsed state to the expanded state.

In a further embodiment of any of the foregoing embodiments, the at least one casing includes a casing port connected to a reservoir in the at least one spring device. The casing port is closed when the at least one casing is in the collapsed state and open when the at least one casing is in the expanded state such that fluid can enter the reservoir from outside of the at least one casing.

In a further embodiment of any of the foregoing embodiments, the reservoir includes an absorbent material.

In a further embodiment of any of the foregoing embodiments, the casing port includes a dissolvable cap.

A further embodiment of any of the foregoing embodiments includes a pass-through conduit extending through the at least one casing, the pass-through conduit includes first and second ends that protrude from the at least one casing.

In a further embodiment of any of the foregoing embodiments, the pass-through conduit includes at least one connector for attaching to an external object.

A further embodiment of any of the foregoing embodiments includes a stent is disposed around the at least one casing. The stent expands when the at least one casing moves from the collapsed state to the expanded state.

A further embodiment of any of the foregoing embodiments includes a driver proximate the at least one casing. The driver has a stored potential energy that is releasable as kinetic energy and, upon release, the driver is expandable to urge the at least one casing to move from a stored position to a deployed position.

In a further embodiment of any of the foregoing embodiments, the at least one casing includes a plurality of casings. The at least one spring device has a plurality of spring devices the at least one inlet port includes a plurality of inlet ports, and the breaker is operable to open the inlet ports and equalize the pressure differentials, whereupon the casings release the spring devices from the stressed state to convert the stored potential energy to kinetic energy that moves the casings from the collapsed state to the expanded state.

In a further embodiment of any of the foregoing embodiments, the at least one casing includes a radiopaque target.

In a further embodiment of any of the foregoing embodiments, the at least one casing incudes a medication that releases when the at least one casing moves from the collapsed state to the expanded state.

A further embodiment of any of the foregoing embodiments includes an absorbent material located on an exterior surface of the at least one casing.

In a further embodiment of any of the foregoing embodiments, the breaker includes a conduit connected with the at least one inlet port, and a pull-cord extending along the conduit and secured with the at least one casing.

In a further embodiment of any of the foregoing embodiments, the pull-cord wraps around the at least one casing such that a pulling force applied to the pull-cord compresses the at least one casing from the expanded state.

In a further embodiment of any of the foregoing embodiments, the casing forms a cup when in the expanded state.

In a further embodiment of any of the foregoing embodiments, an interior of the cup includes an absorbent material.

A further embodiment of any of the foregoing embodiments includes a stopper for limiting movement of the cup.

A further embodiment of any of the foregoing embodiments includes a medicament delivery from at least one casing.

In a further embodiment of any of the foregoing embodiments, at least one casing is released by a dissolvable port in the casing wall.

In a further embodiment of any of the foregoing embodiments, at least one casing includes a radiopaque for location in a body scanner.

A device for expansion in a body cavity according to an example of the present disclosure includes at least one casing that is insertable into the body cavity, the at least one casing has a collapsed state and an expanded state, and at least one spring device in the at least one casing. The at least one casing is evacuated to define a pressure differential that holds the at least one casing in the collapsed state, and in the collapsed state the at least one casing holds the at least one spring device in a stressed state with a stored potential energy. At least one inlet port is connected with the at least one casing. The at least one inlet port is sealed to maintain the pressure differential. A breaker is external to the body cavity when the casing is in the body cavity. The breaker is connected with the at least one inlet port and is operable to open the at least one inlet port and equalize the pressure differential, whereupon the at least one casing releases the at least one spring device from the stressed state to convert the stored potential energy to kinetic energy that moves the at least one casing from the collapsed state to the expanded state that conforms against sides of the body cavity.

A method according to an example of the present disclosure includes providing a device as in any of the foregoing embodiments. The device is inserted into a body cavity, with the breaker external to the body cavity. The breaker is activated by a user to open the at least one inlet port and equalize the pressure differential, whereupon the at least one casing releases the at least one spring device from the stressed state to convert the stored potential energy to kinetic energy that moves the at least one casing from the collapsed state to the expanded state that conforms against sides of the body cavity.

In a further embodiment of any of the foregoing embodiments, the body cavity is selected from the group consisting of a vagina, an anus, an arterial passage, a urinary tract, an ear, a nose, a throat canal, a surgical opening, and a wound.

In a further embodiment of any of the foregoing embodiments, the device further comprises a driver proximate the at least one casing, the driver has a stored potential energy that is releasable as kinetic energy, and the driver is released to urge the at least one casing to move from a stored position at least partially outside of the body cavity to a deployed position in the body cavity.

In a further embodiment of any of the foregoing embodiments, the breaker includes a conduit connected with the at least one inlet port and a pull-cord that extends along the conduit and that is secured with the at least one casing, and a pulling force is applied to the pull-cord to remove the at least one casing from the body cavity.

In a further embodiment of any of the foregoing embodiments, the device further comprises a pass-through conduit that extends through the at least one casing, the pass-through conduit includes first and second ends that protrude from the at least one casing, and an object is passed through the device via the pass-through conduit.

In a further embodiment of any of the foregoing embodiments, in the expanded state the casing plugs the body cavity.

The present disclosure may include any one or more of the individual features disclosed above and/or below alone or in any combination thereof.

### BRIEF DESCRIPTION OF THE DRAWINGS

The various features and advantages of the present disclosure will become apparent to those skilled in the art from the following detailed description. The drawings that accompany the detailed description can be briefly described as follows.

FIG. 1 illustrates an expandable device in a collapsed state.

FIG. 2 illustrates the device of FIG. 1 in an expanded state.

FIG. 3 illustrates an expandable device that has a casing port and reservoir.

FIG. 4 illustrates an expandable device that has a pass-through tube.

FIG. 5 illustrates an expandable device that has a larger pass-through tube.

FIG. 6 illustrates an expandable device with a stent.

FIG. 7 illustrates an expandable device with a driver for deploying the device.

FIG. 8 illustrates the device of FIG. 7 in the deployed position.

FIG. 9A illustrates a device with a cluster of expanders.

FIG. 9B illustrates the cluster of expanders after release.

FIG. 10 illustrates an expandable device with an absorbent material and a pull-cord.

FIG. 11 illustrates an expandable device in which the pull cord wraps around the casing.

FIG. 12 illustrates an expandable device that forms a cup.

FIG. 13 illustrates an expandable device that forms a cup and has a stopper.

FIG. 14A illustrates an expandable device that is configured to expand inwardly to constrict a tube.

FIG. 14B illustrates the device of FIG. 14A in an expanded state.

In this disclosure, like reference numerals designate like elements where appropriate and reference numerals with the addition of one-hundred or multiples thereof designate modified elements that are understood to incorporate the same features and benefits of the corresponding elements.

### DETAILED DESCRIPTION

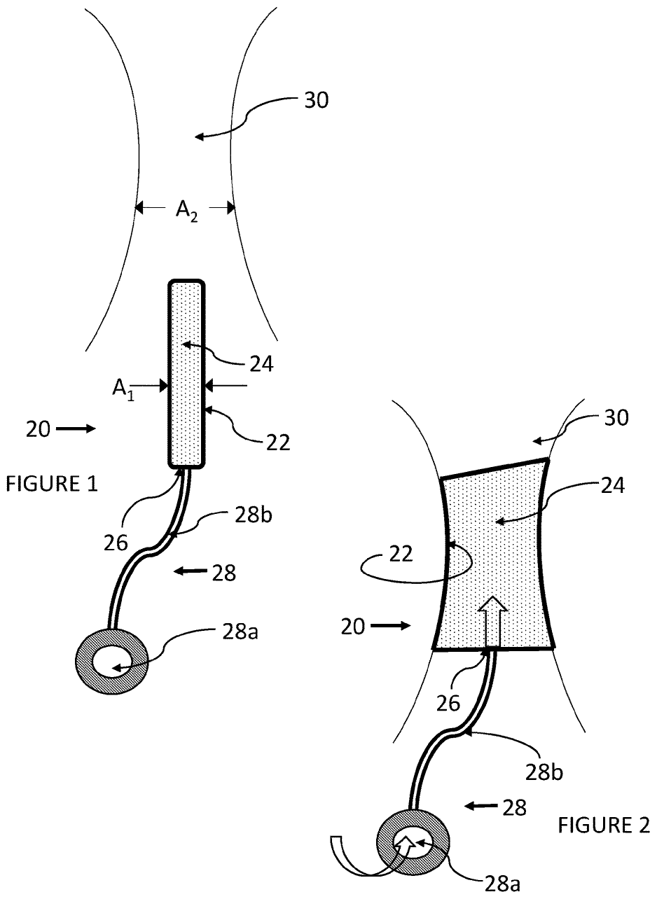

FIG. 1 schematically illustrates an example device 20. As will be appreciated from this and other examples in this disclosure, the device is expandable in a body cavity for such purposes as controlling flow through the cavity, blocking the cavity, or reinforcing the cavity.

The device 20 includes a casing 22 and a spring device 24 in the casing 22. The casing 22 and the spring device 24 together are considered herein to be an expander. The casing 22 may include, but is not limited to, an elastically flexible sack, a convoluted sack, or other mechanical structure that permits the casing to expand/collapse. The casing 22 has a collapsed state (FIG. 1) and an expanded state (FIG. 2). The spring device 24 may be a coil spring, a compressible open-cell foam, a compressible elastic, a compressible textile, a compressible fluid, a collapsible lattice structure, a torsion spring, a constant force spring (e.g., clock spring) or combinations of different types of these or other types of springs, as long as the spring device 24 can be stressed to store potential energy and then elastically recover to release the potential energy as kinetic energy.

The casing 22 is evacuated to define a pressure differential across the spring device 24. The pressure differential holds the casing 22 in the collapsed state. In the collapsed state, the casing 22 holds the spring device 24 in a stressed state having a stored potential energy.

There is an inlet port 26 connected with the casing 22. The inlet port 26 is sealed to maintain the pressure differential across the spring device 24. A breaker 28 is connected with the inlet port 26. For example, the breaker 28 includes a pierceable seal 28a and a conduit 28b connected with the inlet port 26. A breaker tip and button may be used with the pierceable seal 28a to allow a user to pierce the seal 28a and thereby activate the device 20.

The breaker 28 is operable to open the inlet port 26 to equalize the pressure differential. For example, a user activates the breaker 28 by causing the seal 28a to be pierced. Upon venting air through the inlet port 26, the casing 22 releases the spring device 24 from the stressed state to convert the stored potential energy to kinetic energy that moves the casing from the collapsed state to the expanded state as shown in FIG. 2.

In one example of use of the device 20, a user inserts the casing 22 of the device 20 into a body cavity 30 (FIG. 1). The user may be the person of the body cavity or another person, such as a clinician or doctor. As shown, the casing 22 in its collapsed state is smaller in cross-sectional area Al than the cross-sectional area A2 of the body cavity 30. The smaller size permits that casing 22 (and its internal spring device 24) to be inserted into the body cavity. The breaker 28 remains external to the body cavity so that it can be used for activation of the device 20.

Once the casing 22 is inserted into the body cavity 30, the user activates the breaker 28 to open the inlet port 26, permitting air from the surrounding environment to enter through the breaker 28 into the casing 22. The air entering the casing 22 at least partially equalizes the initial vacuum in the casing 22 with the ambient surroundings. Once equalized, or as the vacuum equalizes, the vacuum force holding the casing 22 in its collapsed state decreases and the potential energy of the spring device 24 converts to kinetic energy as the spring device 24 expands. Under the force of expansion of the spring device 24, the casing 22 moves from the collapsed state to the expanded state to take up volume in the body cavity 30. Moreover, as the casing 22 is flexible, it conforms to the shape of the body cavity 30, enabling the now-expanded casing 22 to substantially or completely block the body cavity 30.

The expansion of the spring device 24 increases the volume of the casing 22, which generates a secondary vacuum in the casing 22 that draws an inflow of air from the surrounding environment into the casing 22 to equalize the secondary vacuum. In the figures herein, flow is represented by block arrows. The inflow to equalize this secondary vacuum may be free-flowing or may be controlled via a regulator to thereby control the expansion on the casing 22.

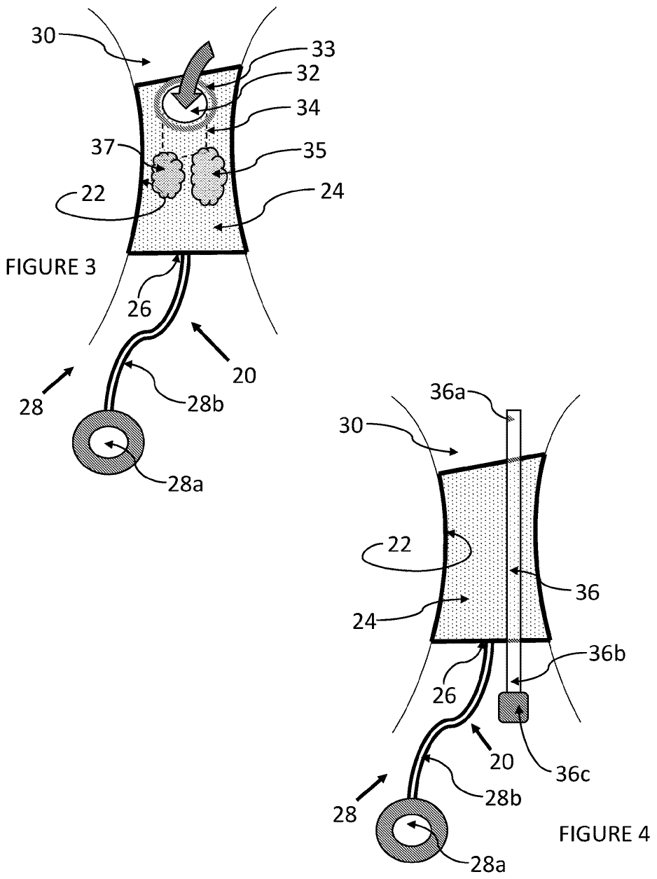

FIG. 3 illustrates a further example of the device 20. In this example, the casing 22 includes a port 32 that is connected to a reservoir 34 in the spring device 24. The port\ 32 is initially closed/collapsed when the casing 22 is in the collapsed state under the pressure differential. The port 32 may be further sealed by an adhesive or elastomer 33, which is then disjoined (e.g., torn) by the expansion of casing 22. When the casing 22 moves to the expanded state, the port 32 opens such that fluid in the body cavity 30 can enter through the port 32 into the reservoir 34 from outside of the casing 22. In embodiments in which the spring device 24 is an absorbent material, such as open cell foam, the fluid moving through port 32 may also be absorbed into and captured by the absorbent material. Either the reservoir 34 or the spring device 24 may include a coagulant 35, for clotting blood, or a superabsorbent polymer, like sodium polyacrylate 37.

FIG. 4 illustrates a further example of the device 20. In this example, the casing 22 includes a pass-through conduit 36, such as a tube, that extends through the casing 22. The pass-through conduit 36 includes first and second ends 36a/36b that protrude from the casing 22. Fluid in the body cavity 30 can flow into and through the first end 36a of the conduit 36 and exit out of the body cavity 30 via the second end 36b of the conduit 36. Thus, although the casing 22 obstructs the body cavity 30, the conduit 36 provides the ability for a controlled amount (by the conduit size) of fluid to pass through the body cavity 30. In a further example, the conduit 36 includes a connector 36c for attaching to an external device, such as a drain bag or suction pump.

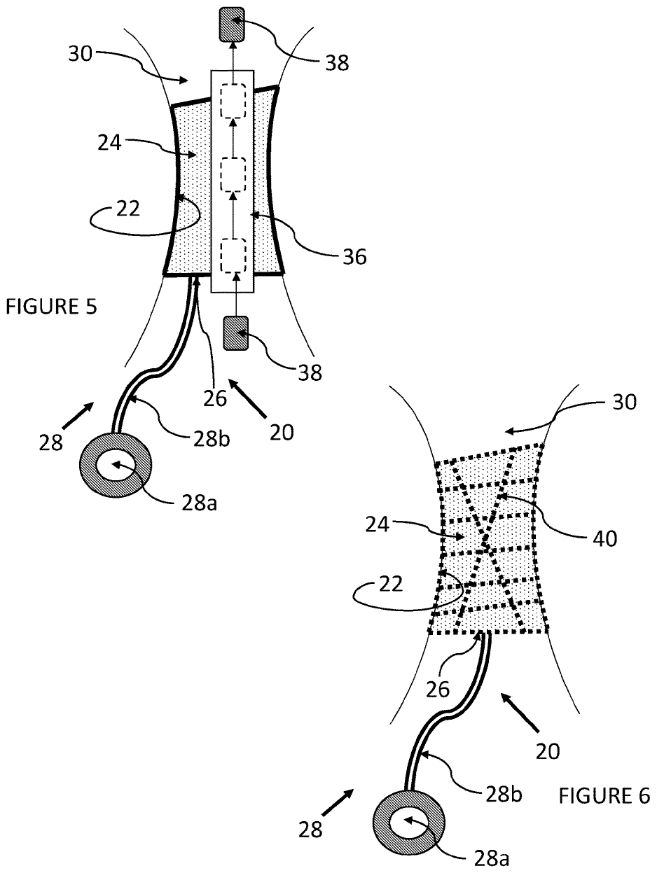

FIG. 5 illustrates a similar example as in FIG. 4, except that the pass-through conduit 36 is larger to enable an object 38, such as an instrument, a medical device, an organ, or medication to be passed through the casing 22 into the body cavity 30.

FIG. 6 illustrates a further example of the device 20. In this example, the device 20 includes a stent 40 disposed around the casing 22. The stent 40, like the casing 22, is initially collapsed but is expandable with the casing 22 as it moves from the collapsed state to the expanded state. For instance, the body cavity 30, such as an artery, may be unstable and the device 20 is used to insert and deploy the stent 40 for cavity stabilization.

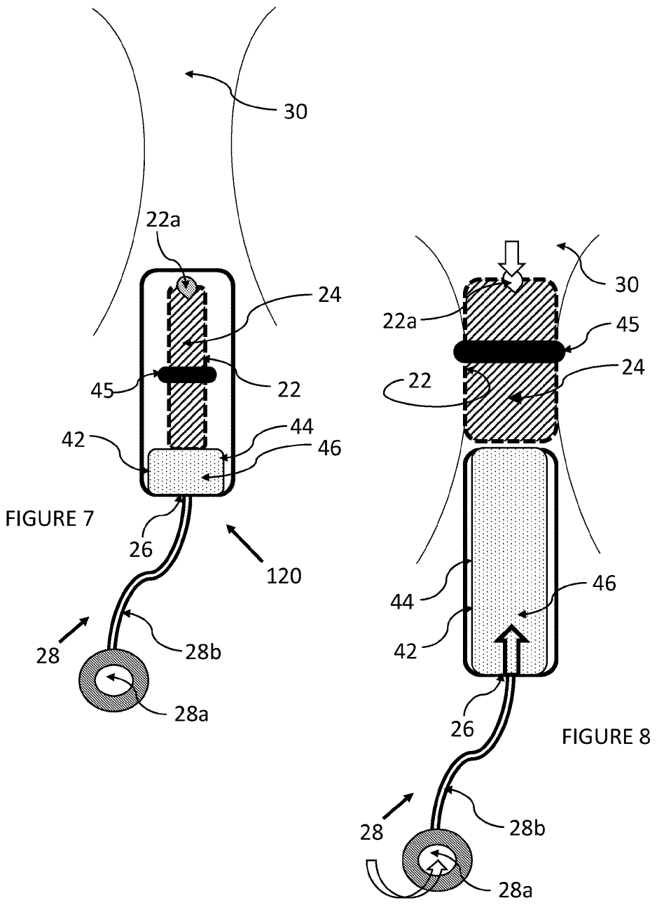

FIG. 7 illustrates another example device 120 that is similar to the device 20 but that additionally includes a driver 42 located proximate the casing 22. The driver 42 has a stored potential energy that is releasable as kinetic energy and, upon release via the breaker 28, the driver 42 is expandable to urge the casing 22 to move from a stored position (FIG. 7) to a deployed position (FIG. 8) at least partially in the body cavity 30. For example, the driver 42 includes a driver casing 44 that is under vacuum to hold a driver spring device 46 in a stressed state. The driver casing 44 is similar to the casing 22 in that it is moveable by the spring device 46 upon opening of the inlet port 26 from a collapsed state to and expanded state. Once released, the driver spring device 46 causes the driver casing 44 to elongate and thereby push the casing 22 at least partially into the body cavity 30, where the spring device 24 also releases to expand the casing 22. The casing 22 may include a dissolvable port cap 22a. The port cap 22a includes a material, for example a polyvinyl alcohol (PVA) or hydroxypropyl methylcellulose (HPMC), that is readily dissolvable in contact with a body fluid, such as blood or urine. Once fully or partially dissolved, the port 22a opens, enabling the venting of air or fluid to equalize the vacuum across the spring device 24. The casing 22 may further include at least one anchor 45 that serves to localize force between the casing 22 and the cavity 30 wall.

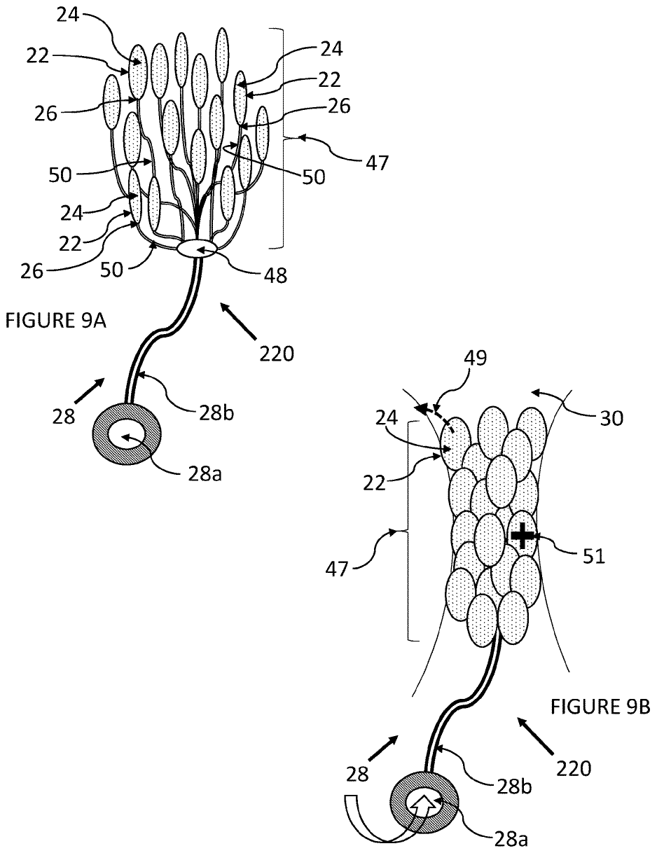

FIG. 9A illustrates another example device 220. In this example, there are a plurality of casings 22 and respective spring devices 24, i.e. a cluster 47 of expanders. In general, the casings 22 are of smaller volume than in prior examples. At splitter 48 the conduit 28b splits into multiple sub-conduits 50 that connect to the inlet ports 26 of the casings 22. Thus, the conduit 28b serves as a common connection such that activation of the breaker 28 serves to release all of the casings 22 and spring devices 24 in unison. For example, the cluster 47 is inserted into the body cavity 30, followed by activation of the breaker 28 to release and expand the cluster of expanders (FIG. 9B) to at least partially fill the body cavity 30. In one embodiment, at least one expander in the cluster 47 includes a substance 49, such as a medication or hydrocolloid. The release and expansion of the expanders in the cluster 47 releases the substance 49 into the body cavity 30, for treatment purposes. In another embodiment, at least one of the expanders includes a radiopaque marker 51 for locating the expander on a body scanning machine, such as an x-ray.

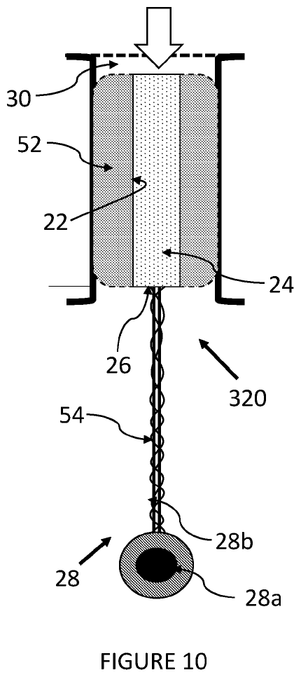

FIG. 10 illustrates another example device 320. In this example, the device 320 additionally includes an absorbent material 52 located on an exterior surface of the casing 22. For example, the absorbent material 52 may include, but is not limited to, cotton, absorbent polymer mesh, or open-cell foam. Upon expansion of the casing 22 to the expanded state (shown), the casing 22 urges the absorbent material 52 against the walls of the body cavity 30 to facilitate absorption of fluid in the cavity 30. The absorbent material 52 may be used to facilitate absorption of an exudate or other substance from the walls of the body cavity 30.

In this example, the device 320 also includes a pull-cord 54 that extends along the conduit 28b. For instance, the pull-cord 54 wraps around the conduit 28b and is attached at one end to the casing 22 and at its opposite end to a portion of the breaker 28 (e.g., a housing). After insertion of the casing 22 into the body cavity 30, the user may apply a pulling force to the pull-cord 54 to remove the casing 22 from the body cavity 30. The conduit 28b may alternatively or additionally be used as a pull-cord for removal, however, the conduit 28b may not be of sufficient strength for reliable removal. In that regard, the pull-cord 54 may be of higher tensile strength to ensure removal. In the illustrated example the pull-cord 54 is wrapped around the outside of the conduit 28b. However, the pull-cord 54 may alternatively be inside the conduit 28b or integrated into the conduit 28b wall.

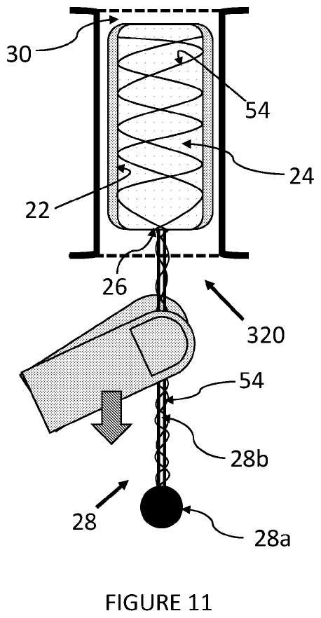

As shown in a further example in FIG. 11, the pull-cord 54 may wrap around the casing 22. When the pulling force is applied to the pull-cord 54, the pull-cord 54 constricts around the casing 22, thereby compressing the casing 22 from the expanded state and enabling easier removal from the body cavity 30. Additionally or alternatively, the pull-cord 54 may act to limit the expansion of the casing 22. In another example, the pull cord 54 is integrated into the wall of the casing 22 or wrapped around the spring device 24 located inside the casing 22.

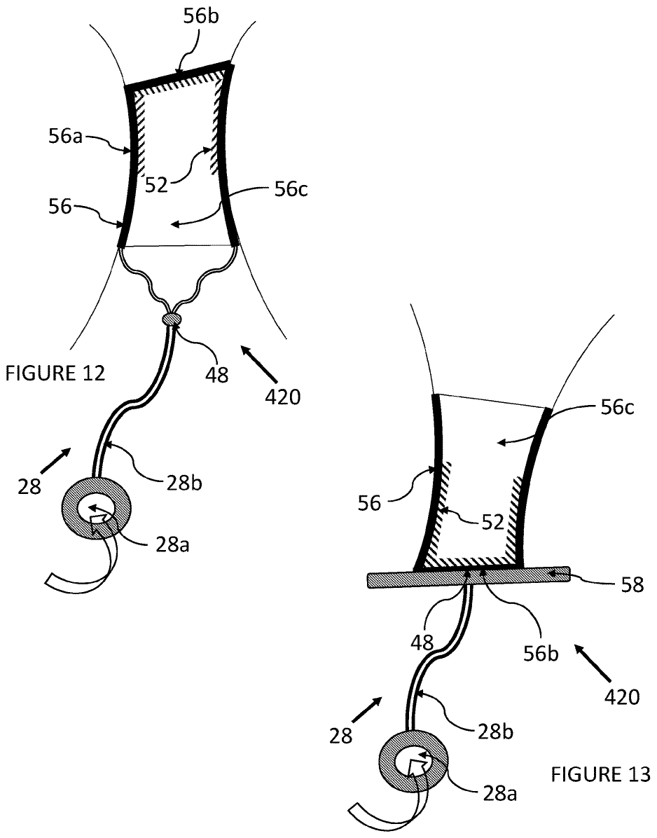

FIG. 12 illustrates another example device 420. The device 420 is similar to the device 20 except that the casing 22 is shaped as an inflatable cup 56. Upon activation to release the spring device 24 in the casing 22, the volume of the casing 22 increases. The increase in volume expands to form an inflatable cup 56, thereby causing the cup to inflate from an initially collapsed state to a deployed state. In the deployed state (shown), the cup 56 includes side walls 56a and a bottom wall 56b that define a cup volume 56c. The top of the cup 56 is open such that fluid in the body cavity 30 collects in the cup volume 56c of the cup 56. As shown, the cup 56 may additionally include an absorbent material 52 in the interior cup volume 56c to soak up the fluid. Depending on the volumetric size of the walls of the cup 56, multiple expanders may be used to achieve full or substantially full inflation.

In the illustrated example, the cup 56 is externally-oriented such that it opens toward the external opening of the body cavity 30 (i.e., outside of the body). In this orientation, the device 420 may be used as a preventative, to catch external fluids coming into the body cavity 30. For example, the device 420 serves as a male or female contraceptive.

In FIG. 13 the device 420 is internally-oriented such that it opens toward the internal opening of the body cavity 30 (i.e., inside of the body). In this orientation, the device 420 may be used as a plug, to catch internal fluids coming out of the body cavity 30. For example, the device 420 serves as a reusable tampon. As also shown in FIG. 13, the device 420 may also include a stopper 58, such as a flange. The stopper 58 is of larger cross-sectional area that the cup 56 in its inflated state such that the stopper 58 catches on the side edges of the body cavity 30 to limit movement of the cup 56 farther into the body cavity 30 once inflated. For example, the device 420 is this orientation may serve as a menstrual cup.

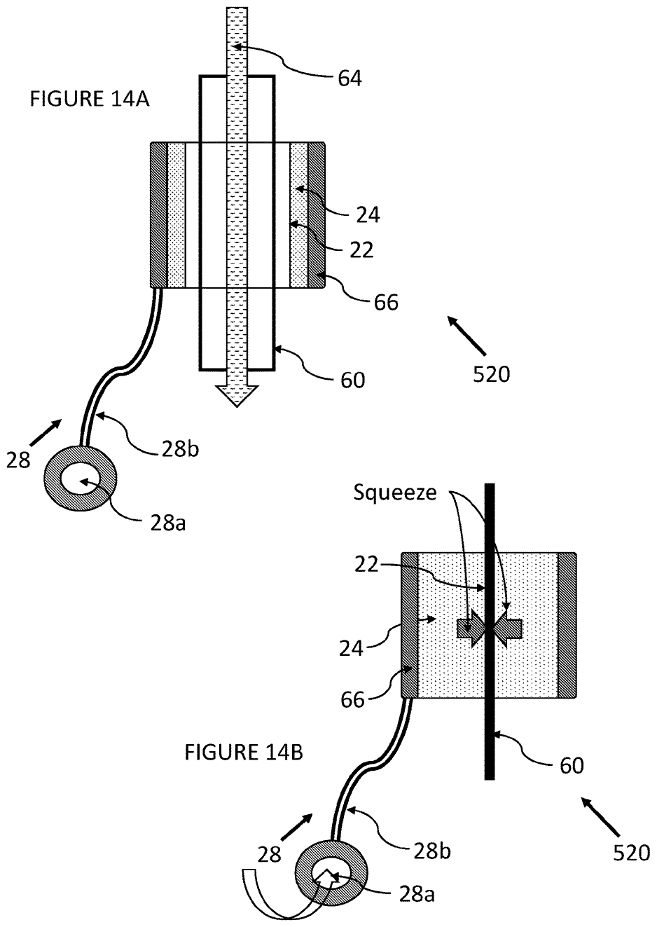

In the prior examples, the movement of the device from the collapsed state to the expanded state was primarily in an outward direction in order to obstruct the body cavity 30. Alternatively, however, a device may be configured for inward expansion, to “squeeze” an object around which the device is arranged. For instance, as shown in FIG. 14A the device 520 is deployed to obstruct flow through a tube 60, which in this case is an artery with blood flow 64. In this example, the casing 22 and spring device 24 are arranged in a shell 66. When the spring device 24 is released, the shell 66 restricts outward expansion of the casing 22 and spring device 24. The casing 22 and spring device 24 thus expand inwardly, i.e., toward the tube 60. The inward expansion pinches the tube 60, thereby constricting the tube 60 and obstructing flow. In one example, the shell 66 is split along its axial direction (relative to the length of the tube 60) to enable the device 520 to be received around the tube 60 without having to fit the device over an end of the tube 60. In a further example, the device 520 can be deflated after by resetting the vacuum in the casing 22 in order to enable the device 520 for another use

As will be appreciated, the devices herein are not limited to hemostasis (blood) control and may be used for facilitation of antibiotic, antiviral, pain remediation (analgesic), ointment, pharmaceutical, or biologic treatments, for absorption and transport of fluids via the open-cell foam, for facilitation of x-ray or magnetic scans, for insertions into small body cavities without use of an external pump such as a syringe-saline pump, for self-adjustment of conformal size to the body cavity without a need for an external control, for addressing multiple blood vessel ruptures within a gunshot wound, and/or for enabling easy removal of an expanded device the end of its useful life, for facilitation of coagulation and platelet activation. In certain configurations the device can be drained, washed, sanitized, sterilized, and reused like with renewable feminine control products for women in resource constrained regions.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

What is claimed is:

1. A device comprising:
at least one casing having a collapsed state and an expanded state;
at least one spring device in the at least one casing, the at least one casing being evacuated to define a pressure differential holding the at least one casing in the collapsed state, and in the collapsed state the at least one casing holding the at least one spring device in a stressed state having a stored potential energy;
at least one inlet port connected with the at least one casing;
a breaker connected with the at least one inlet port, the breaker operable to open the at least one inlet port and equalize the pressure differential, whereupon the at least one casing releases the at least one spring device from the stressed state to convert the stored potential energy to kinetic energy that moves the at least one casing from the collapsed state to the expanded state.

2. The device as recited in claim 1, wherein the at least one casing includes a casing port connected to a reservoir in the at least one spring device, the casing port being closed when the at least one casing is in the collapsed state and open when the at least one casing is in the expanded state such that fluid can enter the reservoir from outside of the at least one casing.

3. The device as recited in claim 2, wherein the reservoir includes an absorbent material.

4. The device as recited in claim 2, wherein the casing port includes a dissolvable cap.

5. The device as recited in claim 1, further comprising a pass-through conduit extending through the at least one casing, the pass-through conduit including first and second ends that protrude from the at least one casing.

6. The device as recited in claim 5, wherein the pass-through conduit includes at least one connector for attaching to an external object.

7. The device as recited in claim 1, further comprising a stent disposed around the at least one casing, the stent expanding when the at least one casing moves from the collapsed state to the expanded state.

8. The device as recited in claim 1, further comprising a driver proximate the at least one casing, the driver having a stored potential energy that is releasable as kinetic energy and, upon release, the driver being expandable to urge the at least one casing to move from a stored position to a deployed position.

9. The device as recited in claim 1, wherein the at least one casing includes a plurality of casings, the at least one spring device includes a plurality of spring devices the at least one inlet port includes a plurality of inlet ports, and the breaker is operable to open the inlet ports and equalize the pressure differentials, whereupon the casings release the spring devices from the stressed state to convert the stored potential energy to kinetic energy that moves the casings from the collapsed state to the expanded state.

10. The device as recited in claim 9, wherein the at least one casing includes a radiopaque target.

11. The device as recited in claim 1, wherein the at least one casing incudes a medication that releases when the at least one casing moves from the collapsed state to the expanded state.

12. The device as recited in claim 1, further comprising an absorbent material located on an exterior surface of the at least one casing.

13. The device as recited in claim 1, wherein the breaker includes a conduit connected with the at least one inlet port, and a pull-cord extending along the conduit and secured with the at least one casing.

14. The device as recited in claim 13, wherein the pull-cord wraps around the at least one casing such that a pulling force applied to the pull-cord compresses the at least one casing from the expanded state.

15. The device as recited in claim 1, wherein the casing forms a cup when in the expanded state.

16. The device as recited in claim 15, wherein an interior of the cup includes an absorbent material.

17. The device as recited in claim 15, further comprising a stopper for limiting movement of the cup.

18. The device as recited in claim 15, further comprising a medicament delivery from at least one casing.

19. The device as recited in claim 15, where at least one casing is released by a dissolvable port in the casing wall.

20. The device as recited in claim 15, where at least one casing includes a radiopaque for location in a body scanner.

21. A device for expansion in a body cavity, the device including at least one casing that is insertable into the body cavity, the at least one casing having a collapsed state and an expanded state, at least one spring device in the at least one casing, the at least one casing being evacuated to define a pressure differential holding the at least one casing in the collapsed state, and in the collapsed state the at least one casing holding the at least one spring device in a stressed state having a stored potential energy, at least one inlet port connected with the at least one casing, the at least one inlet port being sealed to maintain the pressure differential, and a breaker external to the body cavity when the casing is in the body cavity, the breaker connected with the at least one inlet port, the breaker operable to open the at least one inlet port and equalize the pressure differential, whereupon the at least one casing releases the at least one spring device from the stressed state to convert the stored potential energy to kinetic energy that moves the at least one casing from the collapsed state to the expanded state that conforms against sides of the body cavity.

22. A method comprising:
providing a device that comprises at least one casing that has a collapsed state and an expanded state, at least one spring device in the at least one casing, the at least one casing is evacuated to define a pressure differential that holds the at least one casing in the collapsed state, and in the collapsed state the at least one casing holds the at least one spring device in a stressed state that has a stored potential energy, at least one inlet port connected with the at least one casing, the at least one inlet port is sealed to maintain the pressure differential, a breaker connected with the at least one inlet port;
inserting the casing of the device into a body cavity, with the breaker external to the body cavity;
activating the breaker to open the at least one inlet port and equalize the pressure differential, whereupon the at least one casing releases the at least one spring device from the stressed state to convert the stored potential energy to kinetic energy that moves the at least one casing from the collapsed state to the expanded state that conforms against sides of the body cavity.

23. The method as recited in claim 22, wherein the body cavity is selected from the group consisting of a vagina, an anus, an arterial passage, a urinary tract, an ear, a nose, a throat canal, a surgical opening, and a wound.

24. The method as recited in claim 22, wherein the device further comprises a driver proximate the at least one casing, the driver has a stored potential energy that is releasable as kinetic energy, and releasing the driver to urge the at least one casing to move from a stored position at least partially outside of the body cavity to a deployed position in the body cavity.

25. The method as recited in claim 22, wherein the breaker includes a conduit connected with the at least one inlet port and a pull-cord that extends along the conduit and secured with the at least one casing, and applying a pulling force to the pull-cord to remove the at least one casing from the body cavity.

26. The method as recited in claim 22, wherein the device further comprises a pass-through conduit that extends through the at least one casing, the pass-through conduit includes first and second ends that protrude from the at least one casing, and passing an object through the device via the pass-through conduit.

27. The method as recited in claim 22, wherein in the expanded state the casing plugs the body cavity.
